(19) United States
(12) Patent Application Publication
(10) Pub. No.: US 2008/0010755 A1
(43) Pub. Date: Jan. 17, 2008

(54) Use Of Copper Complexes As Light Stabilzers For Textiles

(76) Inventor: Jean-Luc Mura, Rixheim (FR)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/628,531

(22) PCT Filed: May 23, 2005
(86) PCT No.: PCT/IB05/01688

§ 371 Date: Dec. 1, 2006

(30) Foreign Application Priority Data
Jun. 4, 2004 (EP) 04013229.2

(51) Int. Cl. C09K 15/32; C07C 251/24; D06M 13/10; D06M 13/50; D06P 1/62; D06P 1/667; D06P 3/24; D06P 5/02

(52) U.S. Cl. 8/602; 564/269

(57) ABSTRACT

The present invention concerns the use of organic copper complexes based on bisazomethines which, when added as light stabilizers to a dyebath for dyeing textiles, especially undyed polyamide, give rise to much less copper in the dyehouse effluent than substances hitherto used.

The present invention concerns organic copper complexes based on bisazomethines which, when added as light stabilizers to a dyebath for dyeing textiles, especially undyed polyamide, leave behind much less copper in the dyeing effluent than substances hitherto used.

The use of copper salts to improve the light-fastness of textiles is long known. For instance, EP 0 245 204 A1 discloses a process for the photochemical stabilization of dyed and undyed polyamide fibre material or its blends with other fibre materials, wherein the fibre material is treated with a mixture of an organic copper complex, a light stabilizer and if appropriate an antioxidant.

The organic copper complexes previously known and also used in EP 0 245 204 A1, however, have the serious technical disadvantage that too much copper remains in the dyebaths and thus pollutes the effluent. There consequently continues to be a need for environmentally more compatible light stabilizers for textiles.

It has now been found that, surprisingly, certain copper complexes, which are structurally similar to the compounds mentioned above, leave a distinctly smaller amount of copper behind in the dyebath when added as light stabilizers in the dyeing of textiles, and thus are environmentally much more compatible from an ecological viewpoint.

The present invention accordingly provides the use of compounds of the formula (A) 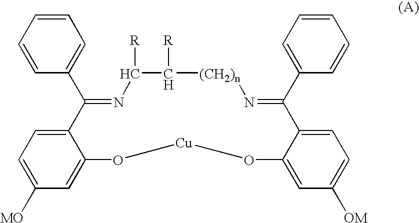
where
n is 0, 1, 2, 3 or 4,
M is H or C₁-C₈-alkyl, and

R is H or where the two R's combine with the carbon atoms to which they are attached to form an aromatic or aliphatic ring having 6 carbon atoms,

as a light-stabilizing addition to the dyebaths for textiles.

Also suitable are compounds wherein n is 0 or 1, preferably 0, and M is H or methyl.

Particularly good results are achieved by using compounds wherein n is 0, M is H or methyl and R is H or where the two R's combine with the carbon atoms to which they are attached to form an aliphatic ring having 6 carbon atoms.

The compound of the formula (I)

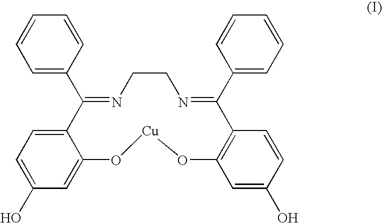

gives especially good results.

The compounds of the formula (II)

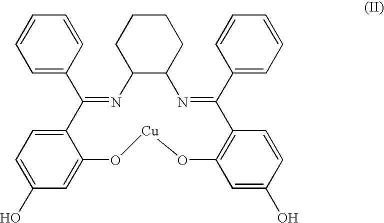

or of the formula (III)

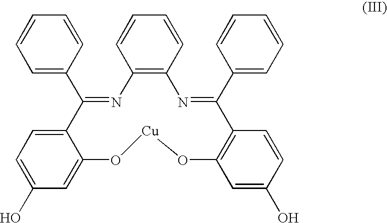

or of the formula (IV)

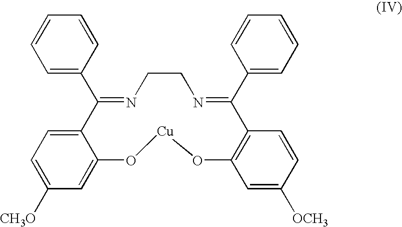

or of the formula (V)

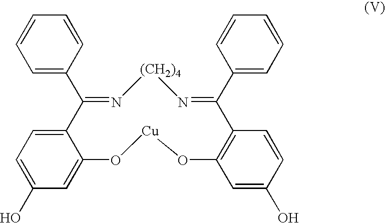

likewise have very good properties when used in the described way.

In the above formulae, the hydrogen atoms in the two —OH groups can be replaced by methyl, as in the formula (IV) for example.

The instant copper complexes are known compounds as disclosed in the following documents:
GUO, YING-CHEN: “Synthesis of N,N′bis(o-hydroxy-p-methoxybenzophenone)-ethylenediamine complexes with copper(II), nickel(II) and cobalt(II)”, STN Database accession no. 2002:403532.
SPIRATOS, MIHAELA ET AL: “Oxygen-carrying polychelates derived from bisphenolic complexes”, STN Database accession no. 1992:165030.
SPIRATOS, MIHAELA ET AL: “Coordination polymers. 7. Synthesis and characterization of some polychelates derived from bisphenolic complexes”, STN Database accession no. 1986:207809.
JENSEN, H. P.: “Structure of copper(II) complexes with Schiff bases derived from reaction of diamines with dihydroxybenzophenone and dihydroxyacetophenone”, STN Database accession no. 1984:521900.
DINJUS, U. ET AL: “Schiff bases of substituted o-hydroxybenzophenones and .alpha.,.omega.-diamines as ligands in 3d-element complexes”, STN Database accession no. 1980:487584.
ZARZHETSKAYA, L. K. ET AL: “Thermostable molding composition”, STN Database accession no. 1976:106625.
U.S. Pat. No. 4,775,386 (BURDESKA KURT ET AL) 4. Oktober 1988 (1988-10-04).

The present compounds are preparable by a process where 2,4-dihydroxybenzophenone or 2-hydroxy-4-methoxybenzophenone is reacted first with a C₂-C₄-alkylenediamine, 1,2-diaminocyclohexane or with o-phenylenediamine and then with a copper salt.

Preferably, 2,4-dihydroxybenzophenone and ethylenediamine are used as starting substances.

The copper compounds of the present invention are suitably used in the form of aqueous dispersions, the concentration of active substance being in the range from 2% to 30% and preferably from 5% to 15% by weight. Dispersions are obtained by grinding in the presence of customary dispersants.

The identified compounds or the dispersions mentioned are very useful as a light-stabilizing additive to textile dyebaths, especially for fibres or wovens of undyed polyamide, the effluent being very much less freighted with copper than in the case of other, commercially available light stabilizers for polyamide, and this constitutes an important technical advantage in view of environmental concerns. This use yields distinct improvements over the prior art for undyed polyamide in particular.

The examples which follow illustrate the invention nonlimitingly.

### EXAMPLES

### Preparation

### Preparation Example 1

In a 750 ml sulphonation flask, 67.55 g of 2,4-dihydroxybenzophenone and 6.1 g of potassium carbonate are mixed in 80 ml of diethylene glycol. This suspension is heated to 80° C., and the 2,4-dihydroxybenzophenone dissolves completely. At 80° C., 9.03 g of ethylenediamine are added over about 10 minutes. After about 30 minutes, the Schiff base starts to precipitate. After 2 hours at 80° C., 150 ml of water are added, the medium is cooled down to room temperature, and the yellow precipitate is filtered off with suction and washed with 200 ml of water. The presscake is dried at 60° C. under reduced pressure to leave 52 g of a yellow powder having the following structure:

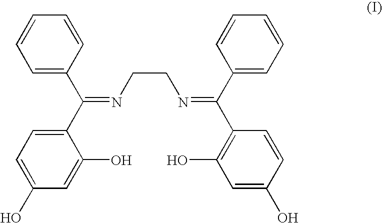

This product is dissolved in 450 ml of dimethylformamide at 100° C. To this solution is added, over 10. minutes, a solution consisting of 28.60 g of copper sulphate pentahydrate, 64 ml of ammonia solution (25%) and 125 ml of water. After about 10 minutes, a pink solid precipitates. After 2 hours under reflux, the batch is cooled down to room temperature, and the precipitate is filtered off and washed with 200 ml of water. The presscake is dried at 60° C. under reduced pressure to leave 47 g of a pinkish violet powder of the formula (I):

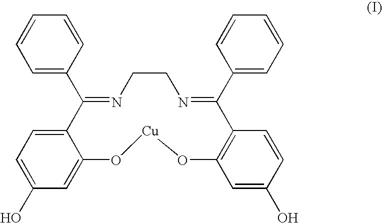

Microanalysis:

| | target | actual |
|---|---|---|
| C | 65.5% | 65.3% |
| H | 4.3% | 4.4% |
| N | 5.4% | 5.3% |
| O | 12.5% | 12.6% |
| Cu | 12.3% | 12.4% |

### Preparation Example 2

In a 750 ml sulphonation flask, 67.55 g of 2,4-dihydroxybenzophenone and 6.1 g of potassium carbonate are mixed in 80 ml of diethylene glycol. This suspension is heated to 80° C., and the 2,4-dihydroxybenzophenone dissolves completely. At 80° C., 17.0 g of 1,2-diaminocyclohexane are added dropwise over about 10 minutes. After about 30 minutes, the Schiff base starts to precipitate. After a further 2 hours at 80° C., 150 ml of water are added, the medium is cooled down to room temperature, and the yellow precipitate is filtered off with suction and washed with 200 ml of water. The presscake is dried at 60° C. under reduced pressure to leave 52 g of a yellow powder having the following structure:

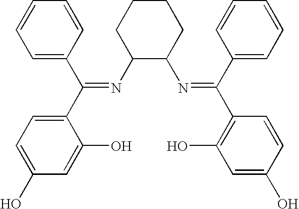

This product is dissolved in 500 ml of dimethylformamide at 100° C. To this solution is added, over 10 minutes, a solution consisting of 25.60 g of copper sulphate pentahydrate, 60 ml of ammonia solution (25%) and 125 ml of water. After about 10 minutes, a dark green solid precipitates. After 2 hours of heating under reflux, the batch is cooled down to room temperature, and the precipitate is filtered off and washed with 200 ml of water. The presscake is dried at 60° C. under reduced pressure to leave 49 g of a dark green powder of the formula (II):

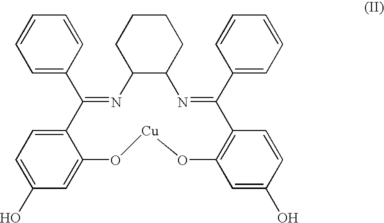

Microanalysis:

| | target | actual |
|---|---|---|
| C | 67.7% | 67.3% |
| H | 4.9% | 5.1% |
| N | 4.9% | 5.0% |
| O | 11.3% | 11.4% |
| Cu | 11.1% | 11.2% |

### Preparation Example 3

In a 750 ml sulphonation flask, 67.55 g of 2,4-dihydroxybenzophenone and 6.1 g of potassium carbonate are mixed in 80 ml of diethylene glycol. This suspension is heated to 80° C., and the 2,4-dihydroxybenzophenone dissolves completely. At 80° C., a solution consisting of 16.2 g of o-phenylenediamine in 100 ml of diethylene glycol is added over about 10 minutes. After about 15 minutes, the Schiff base starts to precipitate. After a further 2 hours at 80° C., 250 ml of water are added, the medium is cooled down to room temperature, and the yellow precipitate is filtered off with suction and washed with 400 ml of water. The presscake is dried at 60° C. under reduced pressure to leave 61 g of a yellow powder having the following structure:

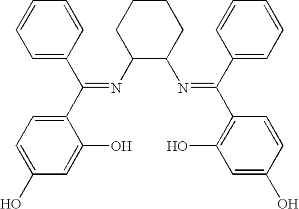

This product is dissolved in 500 ml of dimethylformamide at 100IC. To this solution is added, over 10 minutes, a solution consisting of 30.4 g of copper sulphate pentahydrate, 66 ml of ammonia solution (25%) and 125 ml of water. After about 10 minutes, a dark green solid starts to precipitate. After a further 2 hours under reflux, the batch is cooled down to room temperature, and the precipitate is filtered off and washed with 200 ml of water. The presscake is dried at 60° C. under reduced pressure to leave 59 g of a dark green powder of the formula (III):

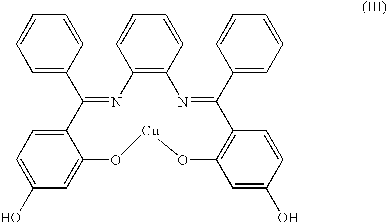

Microanalysis:

| | target | actual |
|---|---|---|
| C | 68.5% | 68.3% |
| H | 3.9% | 4.0% |
| N | 5.0% | 5.1% |
| O | 11.4% | 11.3% |
| Cu | 11.2% | 11.3% |

### Preparation Example 4

In a 750 ml sulphonation flask, 71.96 g of 2-hydroxy-4-methoxybenzophenone and 6.1 g of potassium carbonate are mixed in 80 ml of diethylene glycol. This suspension is heated to 80° C., and the 2-hydroxy-4-methoxybenzophenone dissolves completely. At 80° C., 9.03 g of ethylenediamine are added over about 10 minutes. After about 30 minutes, the Schiff base starts to precipitate. After a further 2 hours at 80° C., 150 ml of water are added, the medium is cooled down to room temperature, and the yellow precipitate is filtered off with suction and washed with 200 ml of water. The presscake is dried at 60° C. under reduced pressure to leave 54 g of a yellow powder having the following structure:

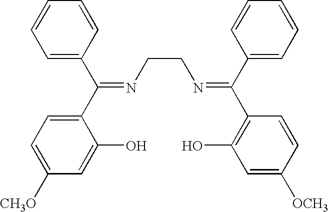

This product is dissolved in 450 ml of dimethylformamide at 100° C. To this solution is added, over 10 minutes, a solution consisting of 28.60 g of copper sulphate pentahydrate, 64 ml of ammonia solution (25%) and 125 ml of water. After about 10 minutes, a pink solid starts to precipitate. After a further 2 hours under reflux, the batch is cooled down to room temperature, and the precipitate is filtered off and washed with 200 ml of water. The presscake is dried at 60° C. under reduced pressure to leave 59 g of a powder of the formula (IV):

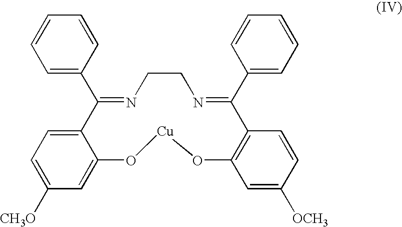

Microanalysis:

| | target | actual |
|---|---|---|
| C | 66.5% | 66.2% |
| H | 4.8% | 5.0% |
| N | 5.2% | 5.1% |
| O | 11.8% | 12.0% |
| Cu | 11.6% | 11.7% |

### Dispersion Example A

### Preparation Example 1

20 parts of copper complex from Preparation Example 1, 20 parts of a sulphonated ditolyl ether-formaldehyde condensate, 10 parts of a triblock copolymer A-B-A (A: polyethylene glycol MW 4700, B: polypropylene glycol MW 2400), 1.2 parts of tridecyl alcohol ethoxylate (9 EO units), 5 parts of polyvinyl alcohol (MW about 67 000), 143.8 parts of demineralized water are ground with 200 parts of glass beads in a dispersing apparatus for 5 hours until the average particle size of the dispersed particles is below 2 micrometers. The glass beads are then separated from the dispersion with the aid of a sieve. The dispersion obtained comprises 10% active.

### Application Example A

100 parts of a nylon 66 carpet from DLW (Deutsche Linoleum Werke, Dietigheim) are introduced into 2000 parts of an aqueous liquor comprising
0.0837 parts of C.I. Acid Orange 80
0.0330 parts of C.I. Acid Black 132
0.0700 parts of C.I. Acid Yellow 235
0.0093 parts of C.I. Acid Violet 90
0.0038 parts of C.I. Acid Red 315
0.0011 parts of C.I. Acid Brown 282

and x parts of the aqueous dispersion prepared according to Dispersion Example A, x being=0, 1, 2, 3 or 4. The pH of the dyebath is adjusted to 6 in the dyeing apparatus, the dyebath is heated to 98° C. over 30 minutes and dyeing is continued at 98° C. for 1 hour. After cooling, the beige dyeing obtained is thoroughly rinsed cold and dried at room temperature. If desired, 1 part of a commercially available levelling agent (for example Sandogen® NH liquid from Clariant) can be added to the liquor. A similar recipe was applied to Nylsuisse nylon and Dura automotive velour from BMW (Bayrische Motorenwerke, Munich, Germany).

The light-fastness according to ISO standard 105-B06 (2, 4 and 6 FAKRA) is measured and the level of residual copper in the dyebaths is determined.

### Comparative Example A

100 parts of a nylon 66 carpet from DLW (Deutsche Linoleum Werke, Dietigheim) are introduced into 2000 parts of an aqueous liquor comprising
0.0837 parts of C.I. Acid Orange 80
0.0330 parts of C.I. Acid Black 132
0.0700 parts of C.I. Acid Yellow 235
0.0093 parts of C.I. Acid Violet 90
0.0038 parts of C.I. Acid Red 315
0.0011 parts of C.I. Acid Brown 282

and x parts of a 10% aqueous dispersion of the following product VP 1: 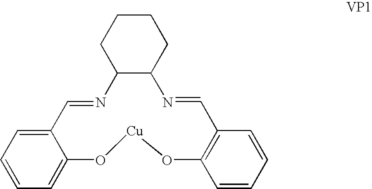
where x is =0, 1, 2 or 3. The pH of the dyebath is adjusted to 6 in the dyeing apparatus, the dyebath is heated to 98° C. over 30 minutes and dyeing is continued at 98° C. for 1 hour. After cooling, the beige dyeing obtained is thoroughly rinsed cold and dried at room temperature. If desired, 1 part of a commercially available levelling agent (for example Sandogen® NH liquid from Clariant) can be added to the liquor. A similar recipe was applied to Nylsuisse nylon and Dura automotive velour from BMW (Bayrische Motorenwerke, Munich, Germany).

The light-fastness according to ISO standard 105-B06 (2, 4 and 6 FAKRA) is measured and the level of residual copper in the dyebaths is determined.

### A RESULTS

| | | Nylsuisse | DLW | BMW |
|---|---|---|---|---|
| | Amount of copper | nylon | carpet | velour |
| | used (ppm in | Residual copper content in | | |
| | bath) | dyebath in ppm | | |
| Application | 6.2 | <1.0 | <1.0 | <1.0 |
| Example A | 12.4 | <1.0 | <1.0 | <1.0 |
| | 18.6 | <1.0 | <1.0 | <1.0 |
| | 24.8 | <1.0 | <1.0 | <1.0 |
| Comparative | 8.4 | <1.0 | <1.0 | <1.0 |
| Example A | 17.7 | 4.0 | 3.0 | 4.0 |
| | 26.0 | 9.0 | 8.0 | 9.0 |

| | Amount of | Amount of | Nylsuisse nylon | | | DLW carpet | | | BMW velour | | |
|---|---|---|---|---|---|---|---|---|---|---|---|
| | copper used | dispersion used | FAKRA | | | | | | | | |
| | (ppm in bath) | (based on nylon) | 2x | 4x | 6x | 2x | 4x | 6x | 2x | 4x | 6x |
| Application | 0 | 0.0% | 1.6 | 1.0 | 1.0 | 1.9 | 1.0 | 1.0 | 1.5 | 1.0 | 1.0 |
| Example A | 6.2 | 1.0% | 4.2 | 2.7 | 2.4 | 3.7 | 3.6 | 3.0 | 4.4 | 2.0 | 1.9 |
| | 12.4 | 2.0% | 4.3 | 3.0 | 2.5 | 4.0 | 4.1 | 3.1 | 4.6 | 3.5 | 3.0 |
| | 18.6 | 3.0% | 4.5 | 3.4 | 2.8 | 4.5 | 4.5 | 4.1 | 4.7 | 3.9 | 3.8 |
| | 24.8 | 4.0% | 4.8 | 3.8 | 3.2 | 4.6 | 4.6 | 4.6 | 4.8 | 4.7 | 4.6 |
| Comparative | 8.4 | 1.0% | 4.3 | 3.1 | 2.7 | 4.0 | 3.3 | 3.0 | 4.6 | 2.7 | 2.7 |
| Example A | 17.7 | 2.0% | 4.4 | 3.5 | 3.1 | 4.6 | 4.3 | 4.2 | 4.5 | 3.3 | 3.3 |
| | 26.0 | 3.0% | 4.3 | 3.8 | 3.4 | 4.6 | 4.5 | 4.4 | 4.5 | 3.9 | 3.4 |

### Application Example B

100 parts of a nylon 66 carpet from DLW (Deutsche Linoleum Werke, Dietigheim) are introduced into 2000 parts of an aqueous liquor comprising
0.1190 parts of C.I. Acid Blue 280
0.1913 parts of C.I. Acid Blue 194
0.0291 parts of C.I. Acid Blue 225
0.0130 parts of C.I. Acid Blue 193
0.0088 parts of.C.I. Acid Blue 296
0.3872 parts of C.I. Acid Black 58
0.1912 parts of CI Acid Black 194

and x parts of the aqueous dispersion prepared according to Dispersion Example A, x being =0, 1, 2, 3 or 4. The pH of the dyebath is adjusted to 6 in the dyeing apparatus, the dyebath is heated to 98° C. over 30 minutes and dyeing is continued at 98° C. for 1 hour. After cooling, the dark blue dyeing obtained is thoroughly rinsed cold and dried at room temperature.

If desired, 1 part of a commercially available levelling agent (for example Sandogen® NH liquid from Clariant) can be added to the liquor. A similar recipe was applied to Nylsuisse nylon and Dura automotive velour from BMW (Bayrische Motorenwerke, Munich, Germany). The light-fastness according to ISO standard 105-B06 (2, 4 and 6 FAKRA) is measured and the level of residual copper in the dyebaths is determined.

### Comparative Example B

100 parts of a nylon 66 carpet from DLW (Deutsche Linoleum Werke, Dietigheim) are introduced into 2000 parts of an aqueous liquor comprising
0.1190 parts of C.I. Acid Blue 280
0.1913 parts of C.I. Acid Blue 194
0.0291 parts of C.I. Acid Blue 225
0.0130 parts of C.I. Acid Blue 193
0.0088 parts of C.I. Acid Blue 296
0.3872 parts of C.I. Acid Black 58
0.1912 parts of CI Acid Black 194

and x parts of a 10% aqueous dispersion of the product VP 1 from Comparative Example A.

The pH of the dyebath is adjusted to 6 in the dyeing apparatus, the dyebath is heated to 98° C. over 30 minutes and dyeing is continued at 98° C. for 1 hour. After cooling, the dark blue dyeing obtained is thoroughly rinsed cold and dried at room temperature. If desired, 1 part of a commercially available levelling agent (for example Sandogen® NH liquid from Clariant) can be added to the liquor. A similar recipe was applied to Nylsuisse nylon and Dura automotive velour from BMW (Bayrische Motorenwerke, Munich, Germany).

The light-fastness according to ISO standard 105-B06 (2, 4 and 6 FAKRA) is measured and the level of residual copper in the dyebaths is determined.

### B RESULTS

| | | Nylsuisse | DLW | BMW |
|---|---|---|---|---|
| | Amount of copper | nylon | carpet | velour |
| | used (ppm in | Residual copper content in | | |
| | bath) | dyebath in ppm | | |
| Application | 6.2 | <1.0 | <1.0 | <1.0 |
| Example B | 12.4 | <1.0 | <1.0 | <1.0 |
| | 18.6 | <1.0 | <1.0 | <1.0 |
| | 24.8 | <1.0 | <1.0 | <1.0 |
| Comparative | 8.4 | <1.0 | <1.0 | <1.0 |
| Example B | 17.7 | 3.0 | 3.0 | 3.0 |
| | 26.0 | 8.0 | 8.0 | 8.0 |

| | Amount of | Amount of | Nylsuisse nylon | | | DLW carpet | | | BMW velour | | |
|---|---|---|---|---|---|---|---|---|---|---|---|
| | copper used | dispersion used | FAKRA | | | | | | | | |
| | (ppm in bath) | (based on nylon) | 2x | 4x | 6x | 2x | 4x | 6x | 2x | 4x | 6x |
| Application | 0 | 0.0% | 2.2 | 1.0 | 1.0 | 3.1 | 2.5 | 1.4 | 3.8 | 2.3 | 1.5 |
| Example B | 6.2 | 1.0% | 3.8 | 1.9 | 1.3 | 3.7 | 3.7 | 3.5 | 4.8 | 3.4 | 3.0 |
| | 12.4 | 2.0% | 4.2 | 2.5 | 1.8 | 4.3 | 4.3 | 3.7 | 4.8 | 4.0 | 4.2 |
| | 18.6 | 3.0% | 4.2 | 2.7 | 2.1 | 4.5 | 4.4 | 3.9 | 4.7 | 4.7 | 4.5 |
| | 24.8 | 4.0% | 4.2 | 3.0 | 2.5 | 4.8 | 4.4 | 4.0 | 4.8 | 4.7 | 4.1 |
| Comparative | 8.4 | 1.0% | 3.8 | 2.1 | 1.6 | 4.2 | 3.5 | 2.5 | 3.8 | 3.7 | 3.6 |
| Example B | 17.7 | 2.0% | 3.9 | 2.5 | 2.0 | 4.9 | 3.7 | 3.5 | 3.8 | 4.2 | 4.3 |
| | 26.0 | 3.0% | 4.1 | 2.7 | 2.2 | 4.9 | 4.4 | 4.1 | 4.1 | 4.2 | 4.2 |

### Application Example C

100 parts of a nylon 66 carpet from DLW (Deutsche Linoleum Werke, Dietigheim) are introduced into 2000 parts of an aqueous liquor comprising
0.0104 parts of C.I. Acid Orange 80
0.0031 parts of C.I. Acid Orange 168
0.0330 parts of C.I. Acid Black 132

and x parts of the aqueous dispersion prepared according to Dispersion Example A, x being =0, 1, 2, 3 or 4. The pH of the dyebath is adjusted to 6 in the dyeing apparatus, the dyebath is heated to 98° C. over 30 minutes and dyeing is continued at 98° C. for 1 hour. After cooling, the grey dyeing obtained is thoroughly rinsed cold and dried at room temperature. If desired, 1 part of a commercially available levelling agent (for example Sandogen® NH liquid from Clariant) can be added to the liquor. A similar recipe was applied to Nylsuisse nylon and Dura automotive velour from BMW (Bayrische Motorenwerke, Munich, Germany).

The light-fastness according to ISO standard 105-B06 (2, 4 and 6 FAKRA) is measured and the level of residual copper in the dyebaths is determined.

### Comparative Example C

100 parts of a nylon 66 carpet from DLW (Deutsche Linoleum Werke, Dietigheim) are introduced into 2000 parts of an aqueous liquor comprising
0.0104 parts of C.I. Acid Orange 80
0.0031 parts of C.I. Acid Orange 168
0.0330 parts of C.I. Acid Black 132

and x parts of a 10% aqueous dispersion of the above used product VP 1. The pH of the dyebath is adjusted to 6 in the dyeing apparatus, the dyebath is heated to 98° C. over 30 minutes and dyeing is continued at 98° C. for 1 hour. After cooling, the grey dyeing obtained is thoroughly rinsed cold and dried at room temperature. If desired, 1 part of a commercially available levelling agent (for example Sandogen® NH liquid from Clariant) can be added to the liquor.

The light-fastness according to ISO standard 105-B06 (2, 4 and 6 FAKRA) is measured and the level of residual copper in the dyebaths is determined.

### C RESULTS

| | | Nylsuisse | DLW | BMW |
|---|---|---|---|---|
| | Amount of copper | nylon | carpet | velour |
| | used (ppm in | Residual copper content in | | |
| | bath) | dyebath in ppm | | |
| Application | 6.2 | <1.0 | <1.0 | <1.0 |
| Example C | 12.4 | <1.0 | <1.0 | <1.0 |
| | 18.6 | <1.0 | <1.0 | <1.0 |
| | 24.8 | <1.0 | <1.0 | <1.0 |
| Comparative | 8.4 | <1.0 | <1.0 | <1.0 |
| Example C | 17.7 | 3.0 | 4.0 | 4.0 |
| | 26.0 | 8.0 | 7.0 | 7.0 |

| | Amount of | Amount of | Nylsuisse nylon | | | DLW carpet | | | BMW velour | | |
|---|---|---|---|---|---|---|---|---|---|---|---|
| | copper used | dispersion used | FAKRA | | | | | | | | |
| | (ppm in bath) | (based on nylon) | 2x | 4x | 6x | 2x | 4x | 6x | 2x | 4x | 6x |
| Application | 0 | 0.0% | 1.1 | 1.0 | 1.0 | 1.7 | 1.0 | 1.0 | 1.0 | 1.0 | 1.0 |
| Example C | 6.2 | 1.0% | 3.8 | 2.4 | 2.2 | 3.8 | 3.2 | 1.7 | 4.1 | 2.0 | 1.4 |
| | 12.4 | 2.0% | 3.8 | 2.4 | 2.4 | 4.0 | 3.3 | 2.2 | 4.5 | 2.4 | 2.0 |
| | 18.6 | 3.0% | 3.9 | 2.6 | 2.5 | 4.4 | 3.8 | 3.0 | 4.6 | 4.0 | 2.8 |
| | 24.8 | 4.0% | 3.8 | 2.9 | 2.6 | 4.8 | 3.8 | 3.5 | 4.6 | 4.0 | 2.8 |
| Comparative | 8.4 | 1.0% | 4.0 | 2.7 | 2.4 | 4.6 | 3.0 | 2.1 | 4.1 | 2.0 | 1.5 |
| Example C | 17.7 | 2.0% | 4.0 | 3.1 | 2.5 | 4.7 | 3.3 | 3.1 | 4.3 | 2.6 | 1.6 |
| | 26.0 | 3.0% | 4.0 | 3.2 | 2.6 | 4.9 | 3.6 | 3.5 | 4.6 | 3.5 | 2.0 |

The present examples show distinctly that the novel compounds, for the same light-fastness, leave a distinctly reduced amount of residual copper behind in the dyebath compared with the prior art, and this constitutes an appreciable technical advantage in view of environmental concerns.

1. A method for stabilizing a dyebath for textiles, said method comprising adding to the dyebath a light-stabilizing additive comprising a compound of the formula (A) or of an aqueous dispersion comprising 2% to 30% by weight of one or more of the compounds of the formula (A) 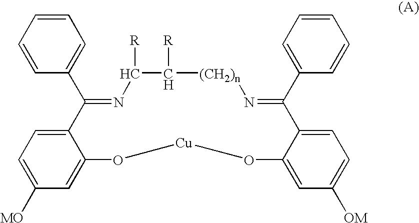
where
n is from 0 to 4,
M is H or C₁-C₈-alkyl, and
R is H or where the two R's combine with the carbon atoms to which they are attached to form an aromatic or aliphatic ring having 6 carbon atoms.

2. The method according to claim 1 wherein
n is 0 or 1, and
M is H or methyl.

3. The method according to claim 1 wherein
n is 0, and
M is H or methyl.

4. The method according to claim 1 wherein
n is 0,
M is H or methyl, and
R is H or where the two R's combine with the carbon atoms to which they are attached to form an aliphatic ring having 6 carbon atoms.

5. The method according to claim 1, wherein formula (A) is formula (I)

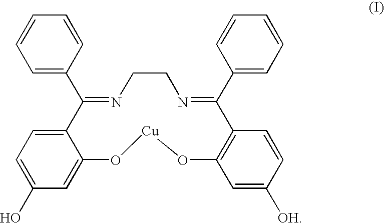

6. The method of claim 1, wherein the aqueous dispersion comprises 5% to 15% by weight of one or more of the compounds of formula (A).

7. The method of claim 1, wherein the textiles are fibres or wovens of undyed polyamide.

8. A light-stabilizing additive for a dyebath for fibers or wovens of undyed polymide textiles, said additive comprising a compound of the formula (A) or an aqueous dispersion comprising 2% to 30% by weight of one or more of the compounds of the formula (A) 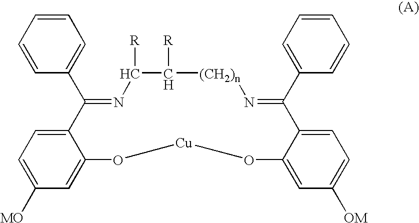
where
n is from 0 to 4,
M is H or C₁-C₈-alkyl, and
R is H or where the two R's combine with the carbon atoms to which they are attached to form an aromatic or aliphatic ring having 6 carbon atoms, whereby the addition of said additive to the dyebath provides light-stabilization to the dyebath.
